(19) United States
(12) Patent Application Publication
(10) Pub. No.: US 2026/0096767 A1
(43) Pub. Date: Apr. 9, 2026

(54) CARDIAC AMBULATORY PATIENT REGISTRATION AND MANAGEMENT PORTAL

(71) Applicant: Bardy Diagnostics, Inc., Bellevue, WA (US)

(72) Inventors: Shawni L. Daw, Redmond, WA (US); Jean D. Cannella, Snoqualmie, WA (US)

(21) Appl. No.: 19/350,596

(22) Filed: Oct. 6, 2025

Related U.S. Application Data
(60) Provisional application No. 63/703,544, filed on Oct. 4, 2024.

(51) Int. Cl. A61B 5/332; A61B 5/00; G16H 40/67

(52) U.S. Cl. CPC A61B 5/332; A61B 5/746; A61B 5/7465; G16H 40/67; A61B 2560/0271

(57) ABSTRACT

A system for electrocardiography monitoring includes a wearable electrocardiography monitoring device and a clinical portal. The clinical portal associates the wearable electrocardiography monitoring device with an individual user. The clinical portal presents (1) patient-specific information and (2) device-specific information to a clinician.

### PRIORITY CLAIM AND CROSS-REFERENCE TO RELATED APPLICATIONS

This application claims priority to, and the benefit of, U.S. Provisional Patent Application No. 63/703,544 , filed Oct. 4, 2024, titled “CARDIAC AMBULATORY PATIETN REGISTRATION AND MANAGEMENT PORTAL,” the entire contents of which are incorporated by reference herein in their entirety and relied upon.

### FIELD

This application relates in general to electrocardiographic monitoring and, in particular, to cardiac ambulatory patient measurement.

### BACKGROUND

The heart emits electrical signals as a by-product of the propagation of the action potentials that trigger depolarization of heart fibers. An electrocardiogram (ECG) measures and records such electrical potentials to visually depict the electrical activity of the heart over time. Conventionally, a standardized set format 12-lead configuration is used by an ECG machine to record cardiac electrical signals from well-established traditional chest locations. Electrodes at the end of each lead are placed on the skin over the anterior thoracic region of the patient's body to the lower right and to the lower left of the sternum, on the left anterior chest, and on the limbs. Sensed cardiac electrical activity is represented by PQRSTU waveforms that can be interpreted post-ECG recordation to derive heart rate and physiology. The P-wave represents atrial electrical activity. The QRSTU components represent ventricular electrical activity.

An ECG is a tool used by physicians to diagnose heart problems and other potential health concerns. An ECG is a snapshot of heart function, typically recorded over 12 seconds, that can help diagnose rate and regularity of heartbeats, effect of drugs or cardiac devices, including pacemakers and implantable cardioverter-defibrillators (ICDs), and whether a patient has heart disease. ECGs are used in-clinic during appointments, and, as a result, are limited to recording only those heart-related aspects present at the time of recording. Sporadic conditions that may not show up during a spot ECG recording require other means to diagnose them. These disorders include fainting or syncope; rhythm disorders, such as tachyarrhythmias and bradyarrhythmias; apneic episodes; and other cardiac and related disorders. Thus, an ECG only provides a partial picture and can be insufficient for complete patient diagnosis of many cardiac disorders.

Diagnostic efficacy can be improved, when appropriate, through the use of long-term extended ECG monitoring. Recording sufficient ECG and related physiology over an extended period is challenging, and often essential to enabling a physician to identify events of potential concern. A 30-day observation period is considered the “gold standard” of ECG monitoring, yet achieving a 30-day observation day period has proven unworkable because such ECG monitoring systems are arduous to employ, cumbersome to the patient, and excessively costly. Ambulatory monitoring in-clinic is implausible and impracticable. Nevertheless, if a patient's ECG could be recorded in an ambulatory setting, thereby allowing the patient to engage in activities of daily living, the chances of acquiring meaningful information and capturing an abnormal event while the patient is engaged in normal activities becomes more likely to be achieved.

For instance, the long-term wear of ECG electrodes is complicated by skin irritation and the inability ECG electrodes to maintain continual skin contact after a day or two. Moreover, time, dirt, moisture, and other environmental contaminants, as well as perspiration, skin oil, and dead skin cells from the patient's body, can get between an ECG electrode, the non-conductive adhesive used to adhere the ECG electrode, and the skin's surface. All of these factors adversely affect electrode adhesion and the quality of cardiac signal recordings. Furthermore, the physical movements of the patient and their clothing impart various compressional, tensile, and torsional forces on the contact point of an ECG electrode, especially over long recording times, and an inflexibly fastened ECG electrode will be prone to becoming dislodged. Moreover, dislodgment may occur unbeknownst to the patient, making the ECG recordings worthless. Further, some patients may have skin that is susceptible to itching or irritation, and the wearing of ECG electrodes can aggravate such skin conditions. Thus, a patient may want or need to periodically remove or replace ECG electrodes during a long-term ECG monitoring period, whether to replace a dislodged electrode, reestablish better adhesion, alleviate itching or irritation, allow for cleansing of the skin, allow for showering and exercise, or for other purpose. Such replacement or slight alteration in electrode location actually facilitates the goal of recording the ECG signal for long periods of time.

Conventionally, Holter monitors are widely used for long-term extended ECG monitoring. Typically, they are used for only 24-48 hours. A typical Holter monitor is a wearable and portable version of an ECG that include cables for each electrode placed on the skin and a separate battery-powered ECG recorder. The cable and electrode combination (or leads) are placed in the anterior thoracic region in a manner similar to what is done with an in-clinic standard ECG machine. The duration of a Holter monitoring recording depends on the sensing and storage capabilities of the monitor, as well as battery life. A “looping” Holter monitor (or event) can operate for a longer period of time by overwriting older ECG tracings, thence “recycling” storage in favor of extended operation, yet at the risk of losing event data. Although capable of extended ECG monitoring, Holter monitors are cumbersome, expensive and typically only available by medical prescription, which limits their usability. Further, the skill required to properly place the electrodes on the patient's chest hinders or precludes a patient from replacing or removing the precordial leads and usually involves moving the patient from the physician office to a specialized center within the hospital or clinic.

The ZIO XT Patch and ZIO Event Card devices, manufactured by iRhythm Tech., Inc., San Francisco, CA, are wearable stick-on monitoring devices that are typically worn on the upper left pectoral region to respectively provide continuous and looping ECG recording. The location is used to simulate surgically implanted monitors. Both of these devices are prescription-only and for single patient use. The ZIO XT Patch device is limited to a 14-day monitoring period, while the electrodes only of the ZIO Event Card device can be worn for up to 30 days. The ZIO XT Patch device combines both electronic recordation components, including battery, and physical electrodes into a unitary assembly that adheres to the patient's skin. The ZIO XT Patch device uses adhesive sufficiently strong to support the weight of both the monitor and the electrodes over an extended period of time and to resist disadherance from the patient's body, albeit at the cost of disallowing removal or relocation during the monitoring period. Moreover, throughout monitoring, the battery is continually depleted and battery capacity can potentially limit overall monitoring duration. The ZIO Event Card device is a form of downsized Holter monitor with a recorder component that must be removed temporarily during baths or other activities that could damage the non-waterproof electronics. Both devices represent compromises between length of wear and quality of ECG monitoring, especially with respect to ease of long term use, female-friendly fit, and quality of atrial (P-wave) signals.

In addition, with the advent of wireless communications and wearable computing, other types of personal ambulatory monitors, of varying degrees of sophistication, have become increasingly available. For example, adherents to the so-called “Quantified Self” movement combine wearable sensors and wearable computing to self-track activities of their daily lives, including inputs, states, and performance. The Nike+ FuelBand, manufactured by Nike Inc., Beaverton, OR, for instance, provides an activity tracker that is worn on the wrist and allows the wearer to temporally track the number of foot steps taken each day and an estimation of the calories burned. The activity tracker can interface with a smart phone device to allow a wearer to monitor their progress towards a fitness goal. Such quantified physiology, however, is typically tracked for only the personal use of the wearer and is not time-correlated to physician-supervised monitoring.

Therefore, a need remains for an extended wear continuously recording ECG monitor practicably capable of being worn for a long period of time in both men and women and capable of recording atrial signals reliably. Moreover, any extended wear continuously recording ECG monitors should be properly registered and associated with individual patients, to ensure adequate tracking and management of measured-data.

### SUMMARY

Physiological monitoring can be provided through a wearable monitor that includes two components, a flexible extended wear electrode patch and a removable reusable monitor recorder. The wearable monitor sits centrally (in the midline) on the patient's chest along the sternum oriented top-to-bottom. The placement of the wearable monitor in a location at the sternal midline (or immediately to either side of the sternum), with its unique narrow “hourglass”-like shape, benefits long-term extended wear by removing the requirement that ECG electrodes be continually placed in the same spots on the skin throughout the monitoring period. Instead, the patient is free to place an electrode patch anywhere within the general region of the sternum, the area most likely to record high quality atrial signals or P-waves. The wearable monitor can also interoperate wirelessly with other wearable physiology and activity sensors and with wearable or mobile communications devices, including so-called “smart phones,” to download monitoring data either in real-time or in batches. The monitor recorder can also be equipped with a wireless transceiver to either provide data or other information to, or receive data or other information from, an interfacing wearable physiology and activity sensor, or wearable or mobile communications devices for relay to a further device, such as a server, analysis, or other purpose.

One embodiment provides a system for electrocardiography monitoring includes a wearable electrocardiography monitoring device and a clinical portal. The clinical portal associates the wearable electrocardiography monitoring device with an individual user. The clinical portal presents (1) patient-specific information and (2) device-specific information to a clinician.

In various embodiments, the clinical portal provides the clinician with a variety of patient-specific information and device-specific information, in an effort to improve device-data management and related efficiencies on a patient-by-patient basis.

In light of the disclosure set forth herein, and without limiting the disclosure in any way, in a first aspect of the present disclosure, which may be combined with any other aspect, or portion thereof, a system for electrocardiography monitoring includes a wearable electrocardiography monitoring device and a clinical portal. The clinical portal associates the wearable electrocardiography monitoring device with an individual user, and wherein the clinical portal presents (1) patient-specific information and (2) device-specific information to a clinician.

In a second aspect of the present disclosure, which may be combined with any other aspect, or portion thereof, the clinician activates the monitoring device via the clinical portal.

In a third aspect of the present disclosure, which may be combined with any other aspect, or portion thereof, the clinician deactivates the monitoring device via the clinical portal.

In a fourth aspect of the present disclosure, which may be combined with any other aspect, or portion thereof, the clinical portal presents the patient-specific information with information for a plurality of additional patients, each associated with a plurality of additional wearable electrocardiography monitoring devices.

In a fifth aspect of the present disclosure, which may be combined with any other aspect, or portion thereof, the clinical portal presents a total wear time, a start date, and end data, and a time remaining for the wearable electrocardiography monitoring device.

In a sixth aspect of the present disclosure, which may be combined with any other aspect, or portion thereof, the clinical portal presents clinician-identified cardiac events and patient-identified cardiac events.

In a seventh aspect of the present disclosure, which may be combined with any other aspect, or portion thereof, the clinical portal presents a device history of device-specific information for a former wearable electrocardiography monitoring device worn prior to the wearable electrocardiography monitoring device.

In an eighth aspect of the present disclosure, which may be combined with any other aspect, or portion thereof, responsive to the wearable electrocardiography monitoring device reaching its total wear time, the clinical portal deactivates the monitoring device.

In a ninth aspect of the present disclosure, which may be combined with any other aspect, or portion thereof, responsive to deactivation of the monitoring device, a deactivation prompt is sent to a mobile device associated with the wearable electrocardiography monitoring device.

In a tenth aspect of the present disclosure, which may be combined with any other aspect, or portion thereof, the clinical portal permits the clinician to view and modify at least one alert threshold for cardiac signals, the alert threshold corresponding to a potential heart problem.

In an eleventh aspect of the present disclosure, which may be combined with any other aspect, or portion thereof, the clinical portal handshakes with the wearable electrocardiography monitoring device, prior to activation of the wearable electrocardiography monitoring device.

In a twelfth aspect of the present disclosure, which may be combined with any other aspect, or portion thereof, the clinician can add doctor-specific notes and associate the doctor-specific notes with the wearable electrocardiography monitoring device.

In a thirteenth aspect of the present disclosure, which may be combined with any other aspect, or portion thereof, the system further includes a mobile device. Cardiac data measured by the wearable electrocardiography monitoring device is transmitted to the mobile device.

In a fourteenth aspect of the present disclosure, which may be combined with any other aspect, or portion thereof, cardiac data measured by the wearable electrocardiography monitoring device is transmitted from the mobile device to an external server.

In a fifteenth aspect of the present disclosure, which may be combined with any other aspect, or portion thereof, cardiac data measured by the wearable electrocardiography monitoring device is accessed at the external server by the clinical portal.

In a sixteenth aspect of the present disclosure, which may be combined with any other aspect, or portion thereof, a system for electrocardiography monitoring includes a wearable electrocardiography monitoring device, a mobile device, and a clinical portal. Cardiac data measured by the wearable electrocardiography monitoring device is transmitted to the mobile device. The clinical portal associates the wearable electrocardiography monitoring device with an individual user.

In a seventeenth aspect of the present disclosure, which may be combined with any other aspect, or portion thereof, the clinical portal presents (1) patient-specific information and (2) device-specific information to a clinician.

In an eighteenth aspect of the present disclosure, which may be combined with any other aspect, or portion thereof, a clinician activates the monitoring device via the clinical portal.

In a nineteenth aspect of the present disclosure, which may be combined with any other aspect, or portion thereof, the clinician deactivates the monitoring device via the clinical portal.

In a twentieth aspect of the present disclosure, which may be combined with any other aspect, or portion thereof, the clinical portal presents a total wear time, a start date, and end data, and a time remaining for the wearable electrocardiography monitoring device.

Still other embodiments will become readily apparent to those skilled in the art from the following detailed description, wherein are described embodiments by way of illustrating the best mode contemplated. As will be realized, other and different embodiments are possible and the embodiments' several details are capable of modifications in various obvious respects, all without departing from their spirit and the scope. Accordingly, the drawings and detailed description are to be regarded as illustrative in nature and not as restrictive.

### BRIEF DESCRIPTION OF THE DRAWINGS

FIGS. 1 and 2 are diagrams showing, by way of examples, an extended wear electrocardiography and physiological sensor monitor respectively fitted to the sternal region of a female patient and a male patient.

FIG. 3 is a functional block diagram showing a system for remote interfacing of an extended wear electrocardiography and physiological sensor monitor in accordance with one embodiment.

FIG. 4 is a perspective view showing an extended wear electrode patch with a monitor recorder inserted.

FIG. 5 is a perspective view showing the monitor recorder of FIG. 4.

FIG. 6 is a perspective view showing the extended wear electrode patch of FIG. 4 without a monitor recorder inserted.

FIG. 7 is a bottom plan view of the monitor recorder of FIG. 4.

FIG. 8 illustrates an exemplary clinical portal, providing various patient data and related device and report statuses.

FIG. 9 illustrates an exemplary clinical portal, providing various report information including availability.

FIG. 10 illustrates an exemplary clinical portal, providing patient-specific device and prescription-related information for an active device.

FIG. 11 illustrates an exemplary clinical portal, providing patient-specific device and prescription-related information for a deactivated device.

FIG. 12 illustrates an exemplary clinical portal, providing patient-specific device and prescription-related information for an incomplete or pre-activated device.

FIG. 13 illustrates an exemplary clinical portal, providing patient-specific device and prescription-related information for a pending or to-be-activated device.

FIG. 14 illustrates an exemplary clinical portal, providing patient-specific device settings for a device.

FIG. 15 illustrates an exemplary clinical portal, providing patient-specific device, report, and event histories.

FIG. 16 illustrates an exemplary clinical portal, providing various patient data and related device and report statuses.

### DETAILED DESCRIPTION

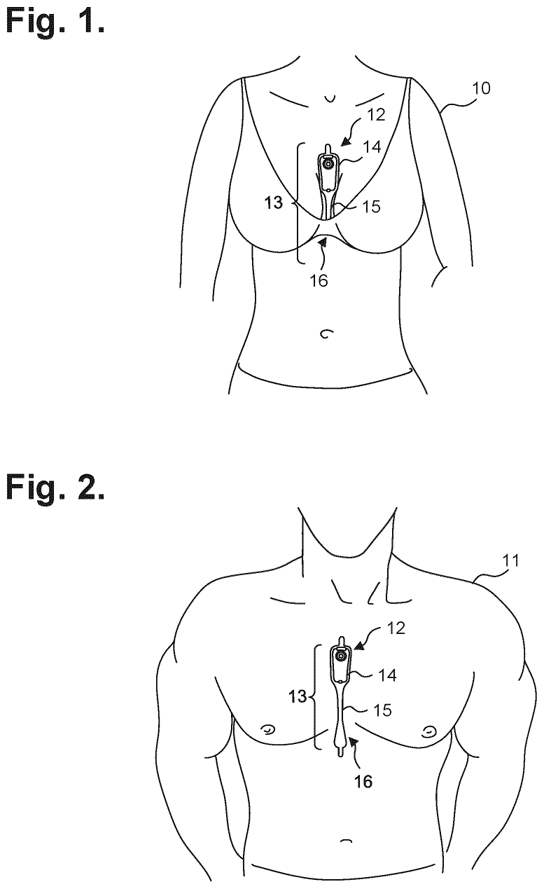

Physiological monitoring can be provided through a wearable monitor that includes two components, a flexible extended wear electrode patch and a removable reusable monitor recorder. FIGS. 1 and 2 are diagrams showing, by way of examples, an extended wear electrocardiography and physiological sensor monitor 12, including a monitor recorder 14 in accordance with one embodiment, respectively fitted to the sternal region of a female patient 10 and a male patient 11. The wearable monitor 12 sits centrally (in the midline) on the patient's chest along the sternum 13 oriented top-to-bottom with the monitor recorder 14 preferably situated towards the patient's head. In a further embodiment, the orientation of the wearable monitor 12 can be corrected post-monitoring, as further described infra. The electrode patch 15 is shaped to fit comfortably and conformal to the contours of the patient's chest approximately centered on the sternal midline 16 (or immediately to either side of the sternum 13). The distal end of the electrode patch 15 extends towards the Xiphoid process and, depending upon the patient's build, may straddle the region over the Xiphoid process. The proximal end of the electrode patch 15, located under the monitor recorder 14, is below the manubrium and, depending upon patient's build, may straddle the region over the manubrium.

The placement of the wearable monitor 12 in a location at the sternal midline 16 (or immediately to either side of the sternum 13) significantly improves the ability of the wearable monitor 12 to cutaneously sense cardiac electric signals, particularly the P-wave (or atrial activity) and, to a lesser extent, the QRS interval signals in the ECG waveforms that indicate ventricular activity, while simultaneously facilitating comfortable long-term wear for many weeks. The sternum 13 overlies the right atrium of the heart and the placement of the wearable monitor 12 in the region of the sternal midline 13 puts the ECG electrodes of the electrode patch 15 in a location better adapted to sensing and recording P-wave signals than other placement locations, say, the upper left pectoral region or lateral thoracic region or the limb leads. In addition, placing the lower or inferior pole (ECG electrode) of the electrode patch 15 over (or near) the Xiphoid process facilitates sensing of ventricular activity and provides superior recordation of the QRS interval.

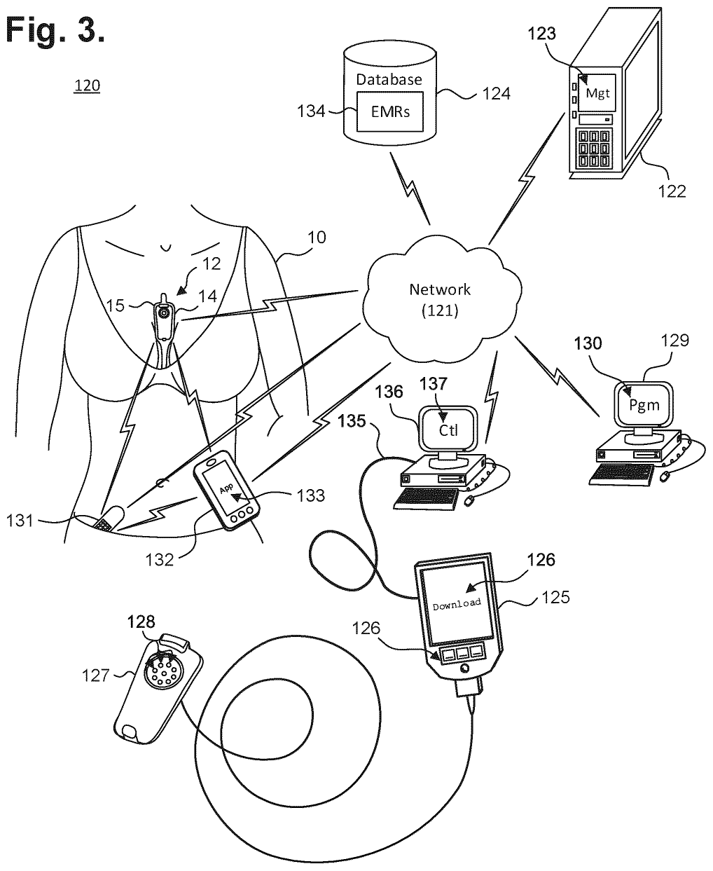

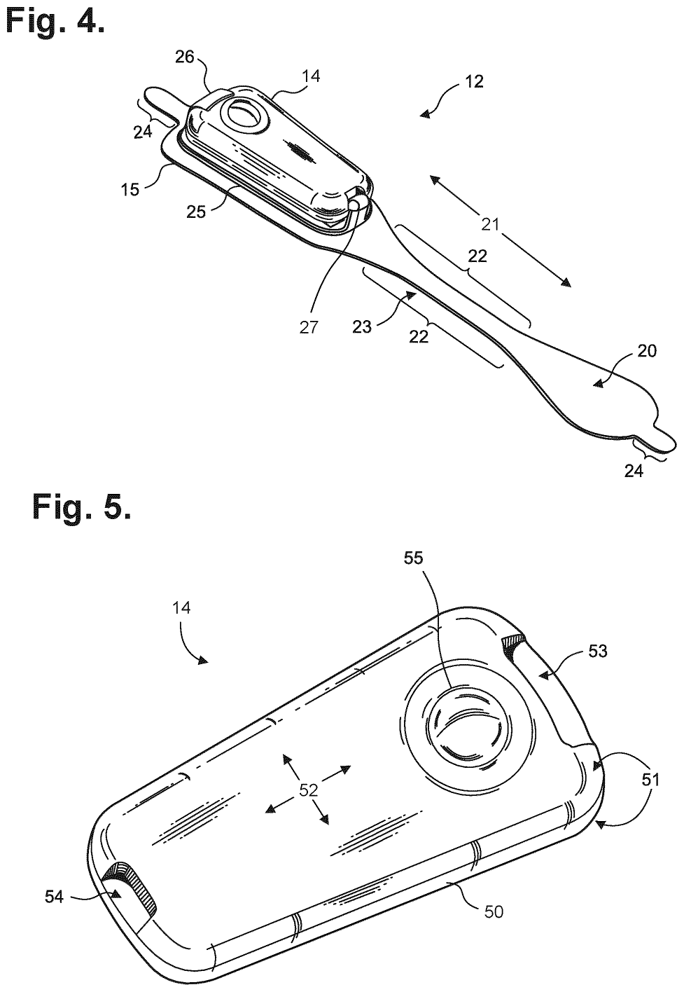

When operated standalone, the monitor recorder 14 of the extended wear electrocardiography and physiological sensor monitor 12 senses and records the patient's ECG data into an onboard memory. In addition, the wearable monitor 12 can interoperate with other devices. FIG. 3 is a functional block diagram showing a system 120 for remote interfacing of an extended wear electrocardiography and physiological sensor monitor 12 in accordance with one embodiment. The monitor recorder 14 is a reusable component that can be fitted during patient monitoring into a non-conductive receptacle provided on the electrode patch 15, as further described infra with reference to FIG. 4, and later removed for offloading of stored ECG data or to receive revised programming. The monitor recorder 14 can then be connected to a download station 125, which could be a programmer or other device that permits the retrieval of stored ECG monitoring data, execution of diagnostics on or programming of the monitor recorder 14, or performance of other functions. The monitor recorder 14 has a set of electrical contacts (not shown) that enable the monitor recorder 14 to physically interface to a set of terminals 128 on a paired receptacle 127 of the download station 125. In turn, the download station 125 executes a communications or offload program 126 (“Offload”) or similar program that interacts with the monitor recorder 14 via the physical interface to retrieve the stored ECG monitoring data. The download station 125 could be a server, personal computer, tablet or handheld computer, smart mobile device, or purpose-built programmer designed specific to the task of interfacing with a monitor recorder 14. Still other forms of download station 125 are possible.

Upon retrieving stored ECG monitoring data from a monitor recorder 14, middleware first operates on the retrieved data to adjust the ECG capture quality, as necessary, and to convert the retrieved data into a format suitable for use by third party post-monitoring analysis software. The formatted data can then be retrieved from the download station 125 over a hard link 135 using a control program 137 (“Ctl”) or analogous application executing on a personal computer 136 or other connectable computing device, via a communications link (not shown), whether wired or wireless, or by physical transfer of storage media (not shown). The personal computer 136 or other connectable device may also execute middleware that converts ECG data and other information into a format suitable for use by a third-party post-monitoring analysis program. Note that formatted data stored on the personal computer 136 would have to be maintained and safeguarded in the same manner as electronic medical records (EMRs) 134 in the secure database 124, as further discussed infra. In a further embodiment, the download station 125 is able to directly interface with other devices over a computer communications network 121, which could be some combination of a local area network and a wide area network, including the Internet, over a wired or wireless connection.

A client-server model could be used to employ a server 122 to remotely interface with the download station 125 over the network 121 and retrieve the formatted data or other information. The server 122 executes a patient management program 123 (“Mgt”) or similar application that stores the retrieved formatted data and other information in a secure database 124 cataloged in that patient's EMRs 134. In addition, the patient management program 123 could manage a subscription service that authorizes a monitor recorder 14 to operate for a set period of time or under pre-defined operational parameters.

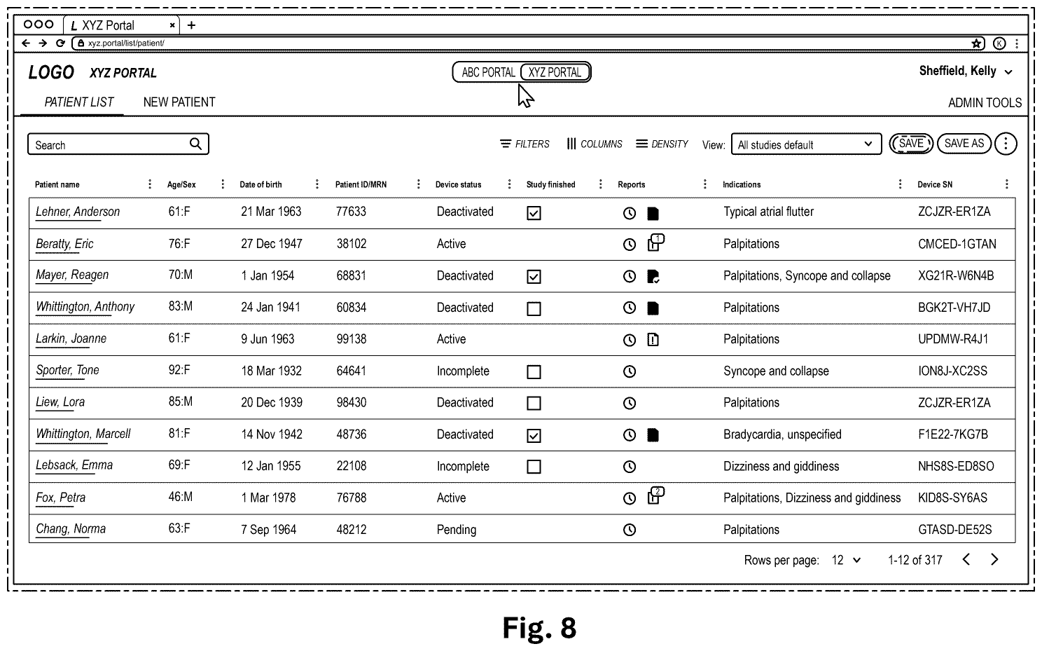

The patient management program 123, or other trusted application, also maintains and safeguards the secure database 124 to limit access to patient EMRs 134 to only authorized parties for appropriate medical or other uses, such as mandated by state or federal law, such as under the Health Insurance Portability and Accountability Act (HIPAA) or per the European Union's Data Protection Directive. For example, a physician may seek to review and evaluate his patient's ECG monitoring data, as securely stored in the secure database 124. The physician would execute an application program 130 (“Pgm”), such as a post-monitoring ECG analysis program, on a personal computer 129 or other connectable computing device, and, through the application 130, coordinate access to his patient's EMRs 134 with the patient management program 123. Other schemes and safeguards to protect and maintain the integrity of patient EMRs 134 are possible. Application 130 may include external clinical portal, as discussed in greater detail herein with reference to FIGS. 8 to 16.

The wearable monitor 12 can interoperate wirelessly with other wearable physiology and activity sensors 131 and with wearable or mobile communications devices 132. Wearable physiology and activity sensors 131 encompass a wide range of wirelessly interconnectable devices that measure or monitor data physical to the patient's body, such as heart rate, temperature, blood pressure, and so forth; physical states, such as movement, sleep, footsteps, and the like; and performance, including calories burned or estimated blood glucose level. These devices originate both within the medical community to sense and record traditional medical physiology that could be useful to a physician in arriving at a patient diagnosis or clinical trajectory, as well as from outside the medical community, from, for instance, sports or lifestyle product companies who seek to educate and assist individuals with self-quantifying interests.

Frequently, wearable physiology and activity sensors 131 are capable of wireless interfacing with wearable or mobile communications devices 132, particularly smart mobile devices, including so-called “smart phones,” to download monitoring data either in real-time or in batches. The wearable or mobile communications device 132 executes an application 133 (“App”) that can retrieve the data collected by the wearable physiology and activity sensor 131 and evaluate the data to generate information of interest to the wearer, such as an estimation of the effectiveness of the wearer's exercise efforts. Still other wearable or mobile communications device 132 functions on the collected data are possible.

The wearable or mobile communications devices 132 could also serve as a conduit for providing the data collected by the wearable physiology and activity sensor 131 to a server 122, or, similarly, the wearable physiology and activity sensor 131 could itself directly provide the collected data to the server 122. The server 122 could then merge the collected data into the wearer's EMRs 134 in the secure database 124, if appropriate (and permissible), or the server 122 could perform an analysis of the collected data, perhaps based by comparison to a population of like wearers of the wearable physiology and activity sensor 131. Still other server 122 functions on the collected data are possible.

During use, the electrode patch 15 is first adhesed to the skin along the sternal midline 16 (or immediately to either side of the sternum 13). A monitor recorder 14 is then snapped into place on the electrode patch 15 to initiate ECG monitoring. FIG. 4 is a perspective view showing an extended wear electrode patch 15 with a monitor recorder 14 in accordance with one embodiment inserted. The body of the electrode patch 15 is preferably constructed using a flexible backing 20 formed as an elongated strip 21 of wrap knit or similar stretchable material with a narrow longitudinal mid-section 23 evenly tapering inward from both sides. A pair of cut-outs 22 between the distal and proximal ends of the electrode patch 15 create a narrow longitudinal midsection 23 or “isthmus” and defines an elongated “hourglass” like shape, when viewed from above.

The electrode patch 15 incorporates features that significantly improve wearability, performance, and patient comfort throughout an extended monitoring period. During wear, the electrode patch 15 is susceptible to pushing, pulling, and torqueing movements, including compressional and torsional forces when the patient bends forward, and tensile and torsional forces when the patient leans backwards. To counter these stress forces, the electrode patch 15 incorporates strain and crimp reliefs, such as described in commonly-assigned U.S. Patent, entitled “Extended Wear Electrocardiography Patch,” U.S. Pat. No. 9,545,204, issued Jan. 17, 2017, the disclosure of which is incorporated by reference. In addition, the cut-outs 22 and longitudinal midsection 23 help minimize interference with and discomfort to breast tissue, particularly in women (and gynecomastic men). The cut-outs 22 and longitudinal midsection 23 further allow better conformity of the electrode patch 15 to sternal bowing and to the narrow isthmus of flat skin that can occur along the bottom of the intermammary cleft between the breasts, especially in buxom women. The cut-outs 22 and longitudinal midsection 23 help the electrode patch 15 fit nicely between a pair of female breasts in the intermammary cleft. Still other shapes, cut-outs and conformities to the electrode patch 15 are possible.

The monitor recorder 14 removably and reusably snaps into an electrically non-conductive receptacle 25 during use. The monitor recorder 14 contains electronic circuitry for recording and storing the patient's electrocardiography as sensed via a pair of ECG electrodes provided on the electrode patch 15, such as described in commonly-assigned U.S. Patent, entitled “Extended Wear Ambulatory Electrocardiography and Physiological Sensor Monitor,” U.S. Pat. No. 9,730,593, issued Aug. 15, 2017, the disclosure which is incorporated by reference. The non-conductive receptacle 25 is provided on the top surface of the flexible backing 20 with a retention catch 26 and tension clip 27 molded into the non-conductive receptacle 25 to conformably receive and securely hold the monitor recorder 14 in place.

The monitor recorder 14 includes a sealed housing that snaps into place in the non-conductive receptacle 25. FIG. 5 is a perspective view showing the monitor recorder 14 of FIG. 4. The sealed housing 50 of the monitor recorder 14 intentionally has a rounded isosceles trapezoidal-like shape 52, when viewed from above, such as described in commonly-assigned U.S. Design Patent, entitled “Electrocardiography Monitor,” No. D717,955, issued Nov. 18, 2014, the disclosure of which is incorporated by reference. The edges 51 along the top and bottom surfaces are rounded for patient comfort. The sealed housing 50 is approximately 47 mm long, 23 mm wide at the widest point, and 7 mm high, excluding a patient-operable tactile-feedback button 55. The sealed housing 50 can be molded out of polycarbonate, ABS, or an alloy of those two materials. The button 55 is waterproof and the button's top outer surface is molded silicon rubber or similar soft pliable material. A retention detent 53 and tension detent 54 are molded along the edges of the top surface of the housing 50 to respectively engage the retention catch 26 and the tension clip 27 molded into non-conductive receptacle 25. Other shapes, features, and conformities of the sealed housing 50 are possible.

The electrode patch 15 is intended to be disposable. The monitor recorder 14, however, is reusable and can be transferred to successive electrode patches 15 to ensure continuity of monitoring. The placement of the wearable monitor 12 in a location at the sternal midline 16 (or immediately to either side of the sternum 13) benefits long-term extended wear by removing the requirement that ECG electrodes be continually placed in the same spots on the skin throughout the monitoring period. Instead, the patient is free to place an electrode patch 15 anywhere within the general region of the sternum 13.

As a result, at any point during ECG monitoring, the patient's skin is able to recover from the wearing of an electrode patch 15, which increases patient comfort and satisfaction, while the monitor recorder 14 ensures ECG monitoring continuity with minimal effort. A monitor recorder 14 is merely unsnapped from a worn out electrode patch 15, the worn out electrode patch 15 is removed from the skin, a new electrode patch 15 is adhered to the skin, possibly in a new spot immediately adjacent to the earlier location, and the same monitor recorder 14 is snapped into the new electrode patch 15 to reinitiate and continue the ECG monitoring.

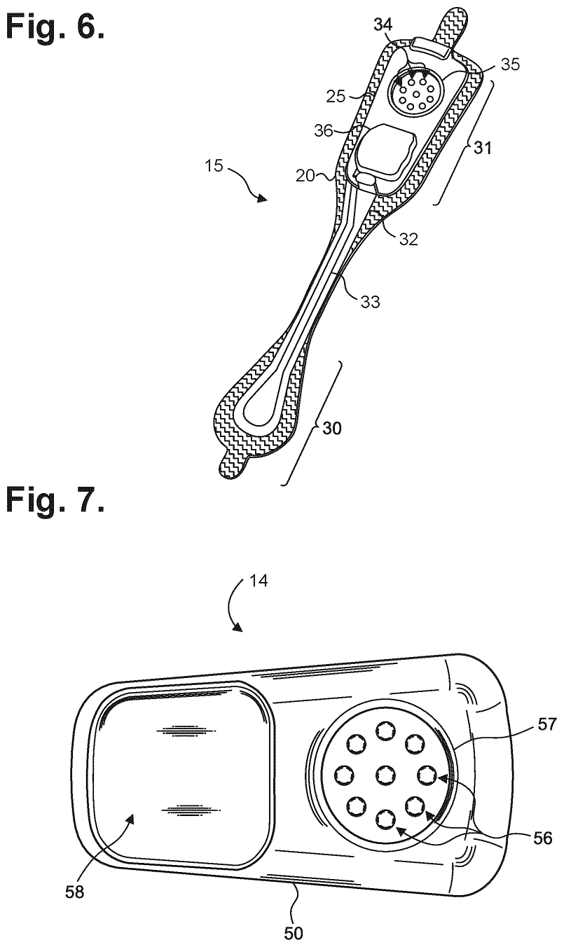

During use, the electrode patch 15 is first adhered to the skin in the sternal region. FIG. 6 is a perspective view showing the extended wear electrode patch 15 of FIG. 4 without a monitor recorder 14 inserted. A flexible circuit 32 is adhered to each end of the flexible backing 20. A distal circuit trace 33 and a proximal circuit trace (not shown) electrically couple ECG electrodes (not shown) to a pair of electrical pads 34. The electrical pads 34 are provided within a moisture-resistant seal 35 formed on the bottom surface of the non-conductive receptacle 25. When the monitor recorder 14 is securely received into the non-conductive receptacle 25, that is, snapped into place, the electrical pads 34 interface to electrical contacts (not shown) protruding from the bottom surface of the monitor recorder 14, and the moisture-resistant seal 35 enables the monitor recorder 14 to be worn at all times, even during bathing or other activities that could expose the monitor recorder 14 to moisture.

In addition, a battery compartment 36 is formed on the bottom surface of the non-conductive receptacle 25, and a pair of battery leads (not shown) electrically interface the battery to another pair of the electrical pads 34. The battery contained within the battery compartment 35 can be replaceable, rechargeable or disposable.

The monitor recorder 14 draws power externally from the battery provided in the non-conductive receptacle 25, thereby uniquely obviating the need for the monitor recorder 14 to carry a dedicated power source. FIG. 7 is a bottom plan view of the monitor recorder 14 of FIG. 4. A cavity 58 is formed on the bottom surface of the sealed housing 50 to accommodate the upward projection of the battery compartment 36 from the bottom surface of the non-conductive receptacle 25, when the monitor recorder 14 is secured in place on the non-conductive receptacle 25. A set of electrical contacts 56 protrude from the bottom surface of the sealed housing 50 and are arranged in alignment with the electrical pads 34 provided on the bottom surface of the non-conductive receptacle 25 to establish electrical connections between the electrode patch 15 and the monitor recorder 14. In addition, a seal coupling 57 circumferentially surrounds the set of electrical contacts 56 and securely mates with the moisture-resistant seal 35 formed on the bottom surface of the non-conductive receptacle 25.

The placement of the flexible backing 20 on the sternal midline 16 (or immediately to either side of the sternum 13) also helps to minimize the side-to-side movement of the wearable monitor 12 in the left- and right-handed directions during wear. To counter the dislodgment of the flexible backing 20 due to compressional and torsional forces, a layer of non-irritating adhesive, such as hydrocolloid, is provided at least partially on the underside, or contact, surface of the flexible backing 20, but only on the distal end 30 and the proximal end 31. As a result, the underside, or contact surface of the longitudinal midsection 23 does not have an adhesive layer and remains free to move relative to the skin. Thus, the longitudinal midsection 23 forms a crimp relief that respectively facilitates compression and twisting of the flexible backing 20 in response to compressional and torsional forces. Other forms of flexible backing crimp reliefs are possible.

As noted at the outset, wearable monitor 12 (and related monitor recorder 14) is associated with an individual patient. Thus, an individual monitor 12 is properly registered and associated with an individual patient, to ensure adequate tracking and management of measured-data. Moreover, an individual mobile communications device 132 is similarly registered and associated with an individual patient. This individual mobile communications device 132 serves as the one-to-one conduit for providing data collected by the wearable monitor 12 to a server 122 and, thus, to a clinician accessing a remote clinical portal.

In an embodiment, each individual monitor 12 is connected to an external clinical portal that allows clinicians to set up the individual monitor 12, register the individual patient (associated with the individual monitor 12), customize monitoring on a patient-by-patient (or device-by-device) basis, and generate data reports.

In an embodiment, the external clinical portal allows allied health professionals to register patients in the portal, order the prescribed device, collect billing/insurance information, determine the device status, locate and find patients from a patient list, locate a report, review a report, provide feedback on the experience, and answer general questions concerning the content and workflows presented in the user interface of the clinical portal.

In an embodiment, the external clinical portal allows healthcare professionals to manage ECG report workflows, find reports, determine report type (e.g., daily, event, summary), add findings to a report, sign-off/approve a summary report, review the report viewer design, provide feedback on the report viewer, and provide feedback on an updated report design (e.g., updated branding, layout and content, and the like).

What follows are screenshots of a proposed external clinical portal, including the related tracking and management of measured data on a patient-by-patient (and device-by-device) basis. It should be appreciated that the graphical layouts presented herein are exemplary only; alternative layouts that convey similar information and provide the user with similar features are, likewise, contemplated herein. The clinician-side access of certain patient and device data, as described in greater detail below, gives the clinician an increased ability to manage a patient set: identifying patient-identified cardiac events, tracking device lifespan for efficient replacement, and registering/activating/deactivating devices to ensure proper patient compliance.

FIG. 8 illustrates an exemplary clinical portal, providing various patient data and related device and report statuses. For example, the portal may provide the clinician with patient information (e.g., name, age/sex, date of birth, and patient ID). The clinical portal further provides the clinician with device information (e.g., device status, study status, and device serial number). Lastly, the clinical portal further provides the clinician with health information, such as data reports and related health indications (e.g., palpitations). Each column of information is sortable. For example, the clinician can sort the patient list by device status, to ensure that the clinician reviews “active” patient listings first, thus avoiding deactivated devices or those that have yet to fully complete registration.

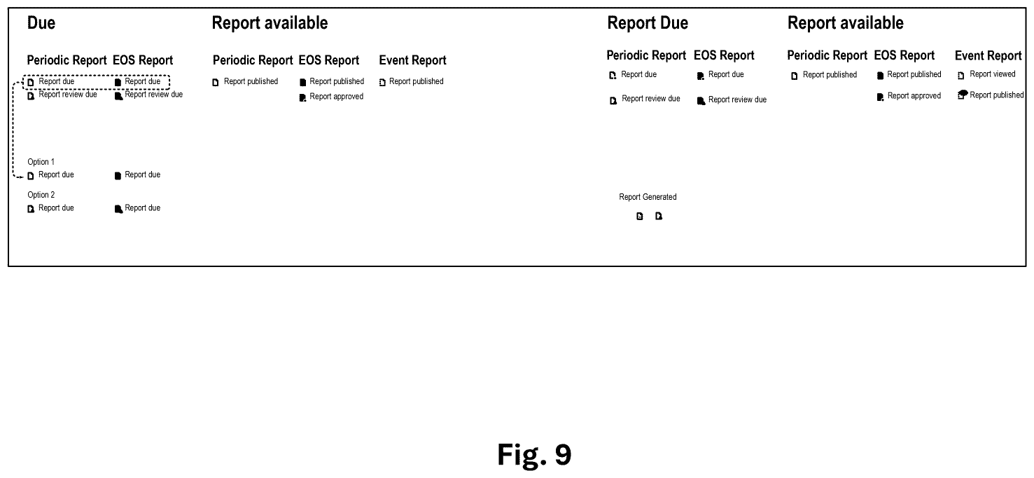

FIG. 9 illustrates an exemplary clinical portal, providing various report information including availability. Report availability can be indicated via report icons. In an embodiment, report icons are used as notifications in the overall patient list, and notify the clinician as to the status, type, and availability of respective reports.

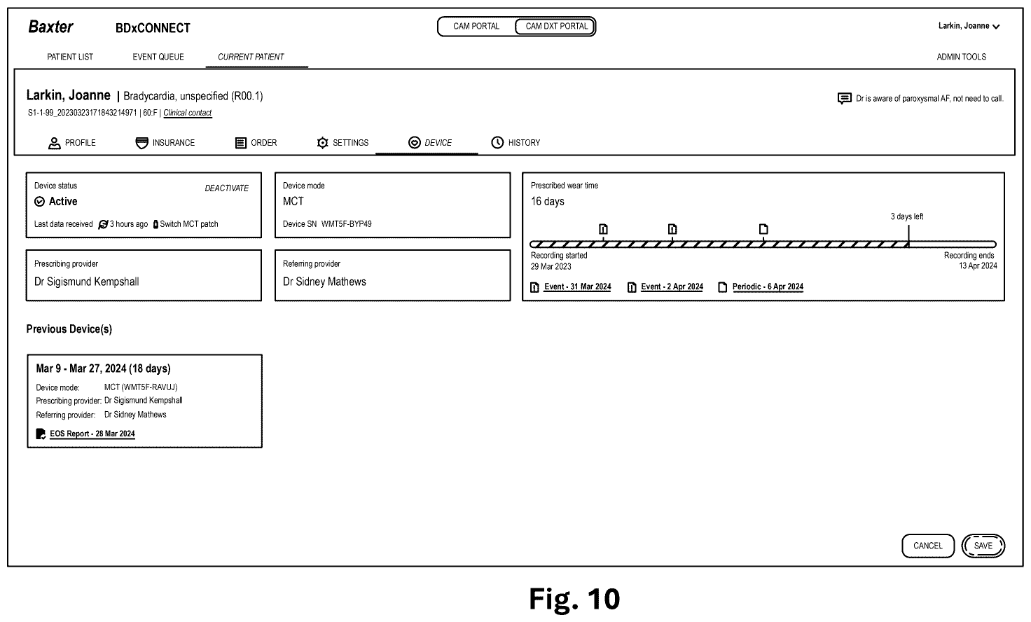

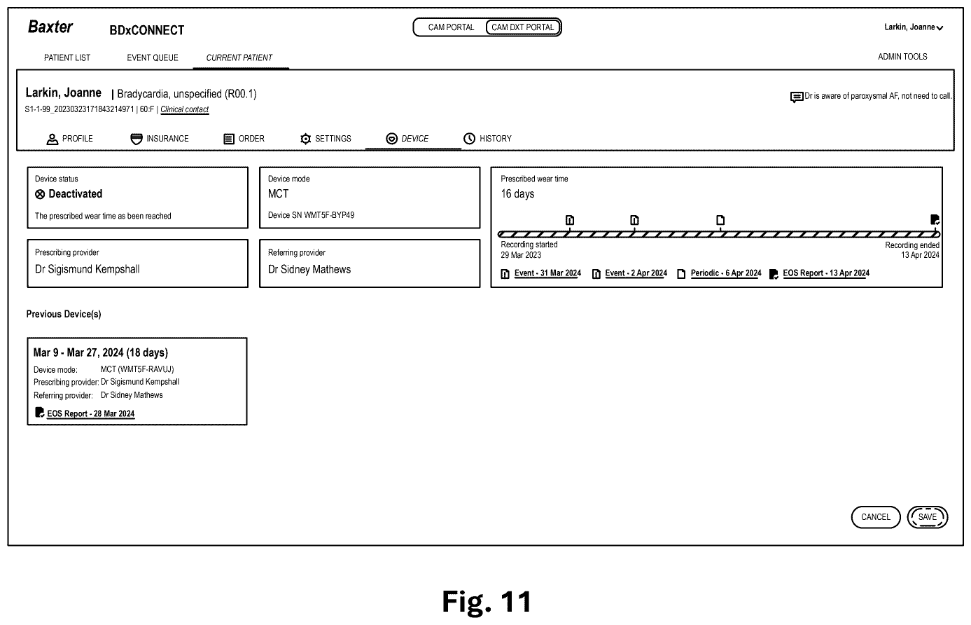

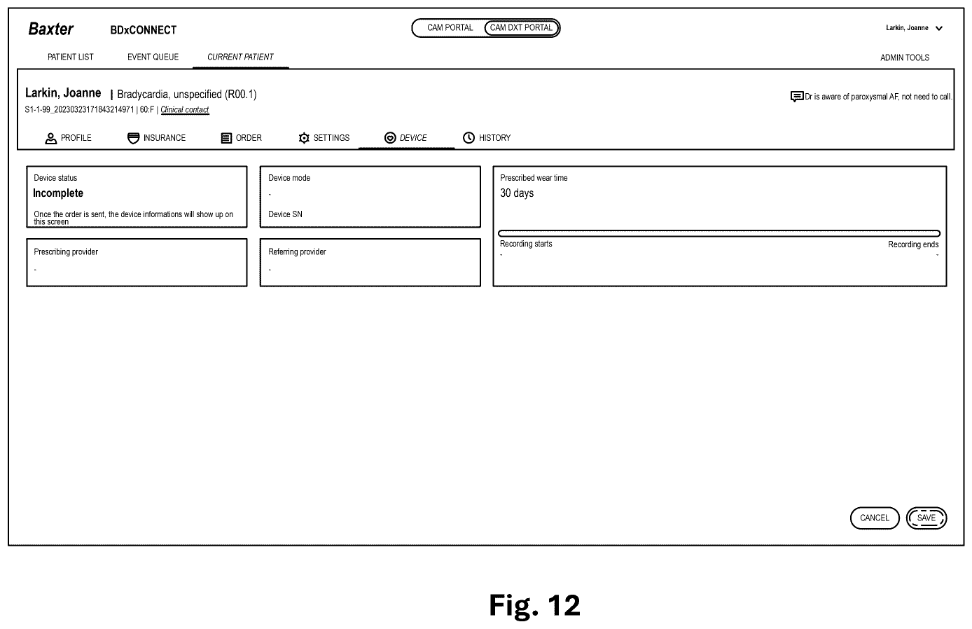

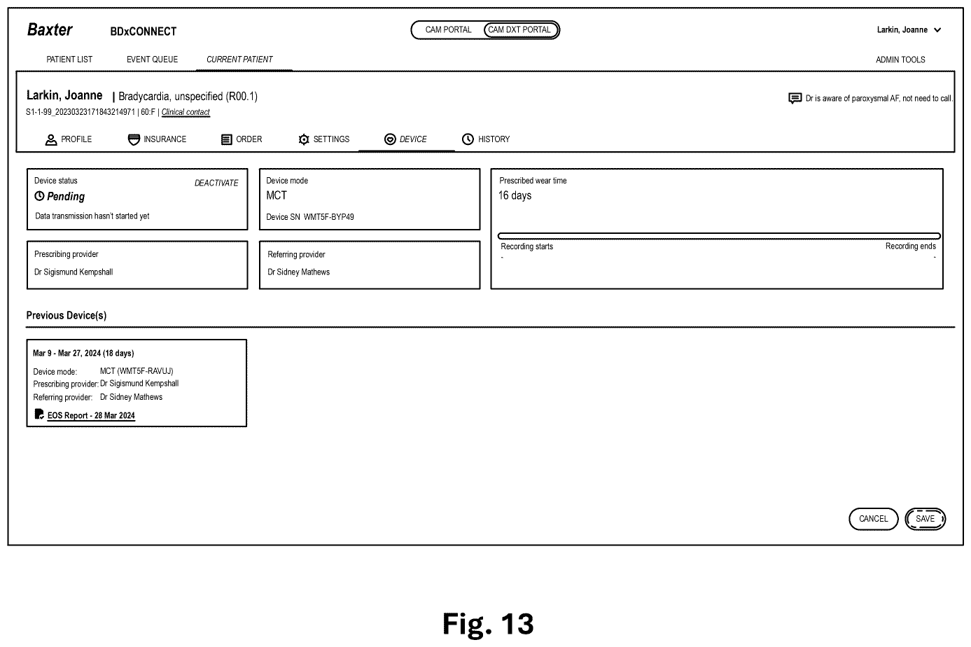

FIGS. 10 to 13 illustrate an exemplary clinical portal, including a device page for a specific patient. In an embodiment, the device page provides a dashboard design displaying the status of the device. For example, as illustrated in FIG. 10, the device is indicated as active. As illustrated in FIG. 11, the device is indicated as deactivated. As illustrated in FIG. 12, the device is indicated as incomplete. As illustrated in FIG. 13, the device is indicated as pending. The dashboard design may additional display an activity timeline and related events, as features via the use of an icon.

More specifically, FIG. 10 illustrates an exemplary clinical portal, providing patient-specific device and prescription-related information for an active device. The portal provides patient-doctor information, including both the prescribing and referring providers. This is helpful for clinical-side data processing, particularly when it comes to insurance reimbursement and related administrative processing. FIG. 10 shows the prescribed wear time for the device (e.g., wearable monitor 12), including a prescribed total wear time, a start date, and end date, a time remaining (e.g., “three days left”), and a graphical depiction of time remaining. Along the graphical depiction of the device wear-time lists various events, including both clinician-identified cardiac events and patient-identified cardiac events; the different event types are presented graphically as different icons. This helps the clinician distinguish algorithm/clinician-identified cardiac events from patient-identified cardiac events, and thus prioritize different types of events accordingly. FIG. 10 further includes a previous device history, including a devices wear-time and related information for that device. Reports on that device (i.e., the former device) are selectable by the clinician. This allows for the clinician to dig into past data on a device-by-device basis, to identify correlation across multiple devices and/or identify device anomalies if there are certain device-specific outliers.

FIG. 11 illustrates an exemplary clinical portal, providing patient-specific device and prescription-related information for a deactivated device. FIG. 11 shows the prescribed wear time for the device (e.g., wearable monitor 12), including a prescribed total wear time, a start date, and end date, and a graphical depiction of time remaining. Given wearable monitor 12 has reached its prescribed wear time, as illustrated, monitor 12 is subsequently deactivated. In an embodiment, a deactivation prompt is sent to mobile communications device 132. Deactivating monitor 12 when it reaches its end of life ensures that the device does not record (and the clinician does not subsequently analyze/process) data that could be faulty (e.g., due to patch failure and adhesive loss, due to reduced signal acquisition via battery degradation, and the like). In an embodiment, when monitor 12 reaches its end of life, mobile communications device 132 provides the patient with a link providing step-by-step instructions on how to physically return monitor 12. In an embodiment, as monitor 12 nears its end of life (e.g., 24 hours before end of life) a notification is sent to the mobile communications device 132 to remind the patient to replace the monitor 12 with a new monitor 12. Likewise, in an embodiment, as monitor 12 nears full memory (e.g., 90% of memory storage full) a notification is sent to the mobile communications device 132 to remind the patient to replace the monitor 12 with a new monitor 12 and/or ensure proper data transmission to mobile communications device 132 to offload memory.

FIG. 12 illustrates an exemplary clinical portal, providing patient-specific device and prescription-related information for an incomplete or pre-activated device. Namely, until the patient properly activates the wearable monitor 12, going through several steps for physical application, device setup, device handshaking with mobile communications device 132, and the like, the wearable monitor 12 will not provide data to the clinical portal. Each of these activation steps (e.g., physical application of the patch onto the patient's sternum) may be provided via step-by-step instruction for the patient at the mobile communications device 132. Until the patient completes the step-by-step instruction, the monitor 12 will not be activated. Even if monitor 12 is recording and sending data, the data will be disregarded until the wearable monitor 12 is fully set up as a complete and activated device. In an embodiment, monitor 12 requires a certain quantity of received data (e.g., baseline readings) prior to activation, to ensure that the measured signal is of sufficient quality for subsequent monitoring.

FIG. 13 illustrates an exemplary clinical portal, providing patient-specific device and prescription-related information for a pending or to-be-activated device. Namely, in certain embodiment, even after the patient takes the required steps to properly activate wearable monitor 12, the device requires clinician-side authorization and/or handshaking before the device is activated to begin recording/transmitting data. As an example, the clinician can wait until the device is fully approved by insurance (e.g., for reimbursement) before “approving” the device and permitting it to operate in an active state. Remote activation/deactivation, via the clinician side, ensures a more efficient device-data protocol, as data is not recorded and transmitted until the device is permitted to do so. Moreover, the clinician's ability to remotely active/deactivate allows the clinician to consider third-party inputs, such as the patient's referring provider, prescribing provider, insurance company, and the like.

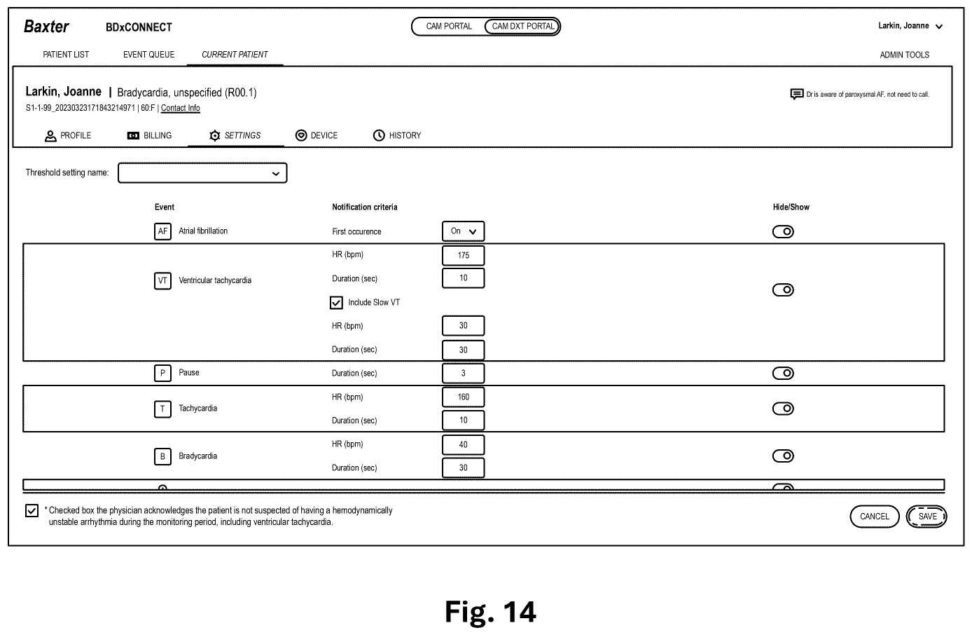

FIG. 14 illustrates an exemplary clinical portal, providing patient-specific device settings for wearable monitor 12 including threshold settings. In an embodiment, providers can adjust the settings at any point. For example, providers have the option of viewing, or not viewing, slow ventricular tachycardia. The clinician is able to implement thresholds for individual patients wearing individual devices, ensuring a much greater degree of control over the data flow into the portal. Notifications can be triggered for atrial fibrillation at first occurrence, for ventricular tachycardia when heart rate exceeds a pre-set beats-per-minute for a particular duration, when data pauses for a pre-set time period, for tachycardia when heart rate exceeds a pre-set beats-per-minute for a particular duration, and for bradycardia when heart rate exceeds a pre-set beats-per-minute for a particular duration. Advantageously, as noted previously, this permits a clinician to “dial in” the settings as a patient is continually monitored, particularly across multiple patches. For example, if a patient has several tachycardia events with a first wearable monitor 12, when the patient applies the second wearable monitor 12 the clinician may want to lower the threshold for triggering tachycardia (as they are more likely to occur given recent data) and perhaps “turn off” certain other alerts (or turn them down) so as to ensure that the second wearable monitor 12 is more fine-tuned for tachycardia identification.

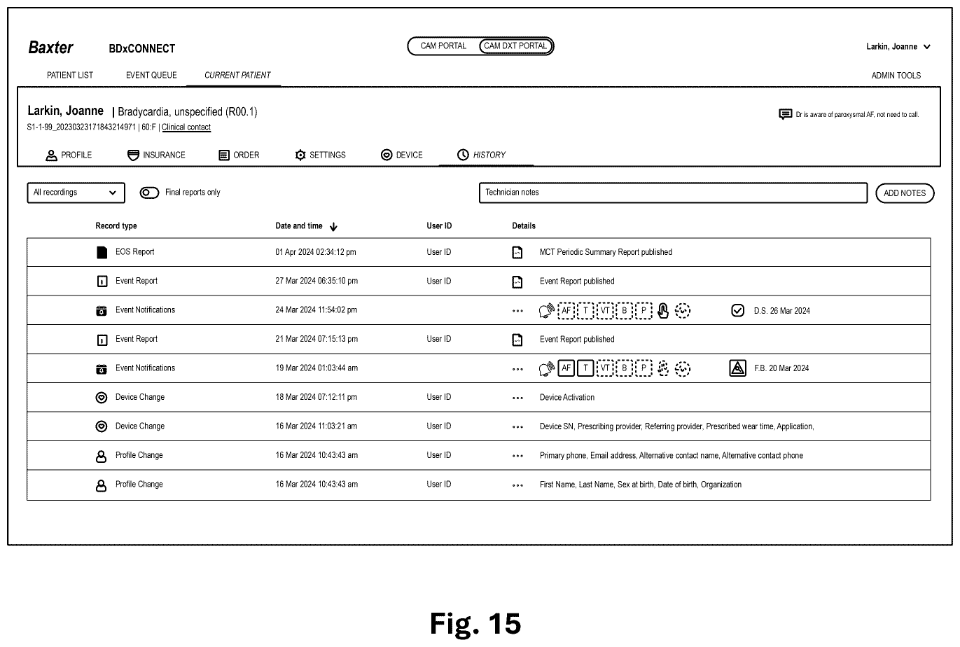

FIG. 15 illustrates an exemplary clinical portal, providing patient-specific device, report, and event histories. In an embodiment, patient history includes patient events and compiles key points in the timeline such as any changes to settings, patient profile, device, event notifications, event report, and other related report types. Advantageously, the clinician can sort this information by date/time when the change was made.

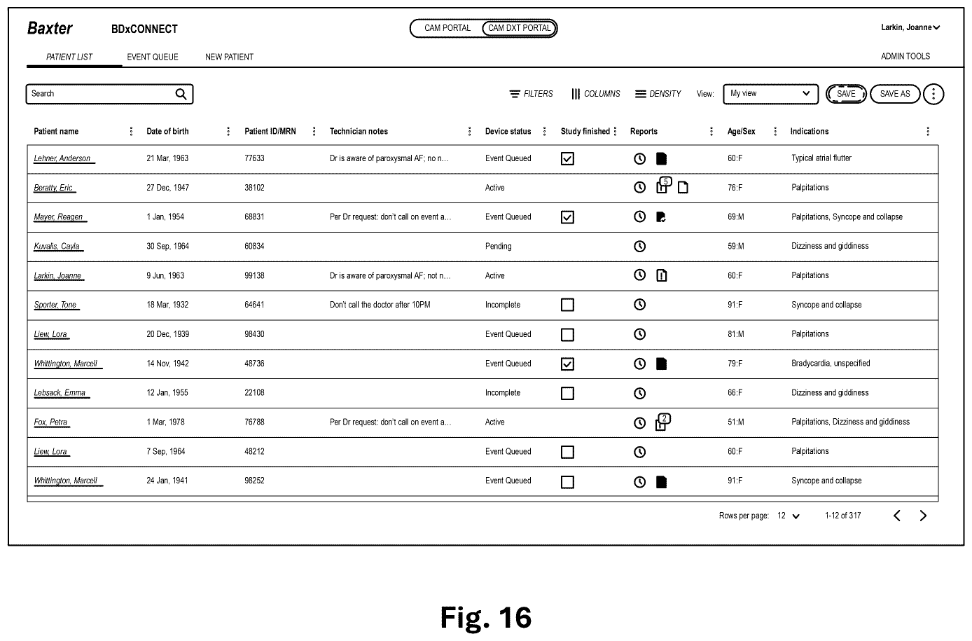

FIG. 16 illustrates an exemplary clinical portal, providing various patient data and related device and report statuses. Similar to FIG. 8, the portal in FIG. 16 may provide the clinician with patient information (e.g., name, date of birth, and patient ID). The clinical portal further provides the clinician with device information (e.g., device status and study status). Lastly, the clinical portal further provides the clinician with health information, such as data reports and related health indications (e.g., palpitations). The portal in FIG. 16 further provides the clinician with technician notes. For example, an individual patient's doctor may indicate that he or she does not want to be called after 10 PM in the evening if there's a cardiac event. As another example, an individual patient's doctor may already be aware of certain cardiac issues, and will not want to be needlessly alerted when those events are identified for the first time via the clinical portal (as they have been identified previously by the doctor in other settings).

In an embodiment, the patient list is the listing of current patients. The list also allows for addition of new patients, and a launching point into any individual patient's landing page, where the profile, insurance, order, settings, device and history can be accessed. In an example, the “Event Queue” is only viewable in the ECG technician view. In other words, various views are displayed (and controller) by one's role in an organization; for example, healthcare professionals and allied health professionals may not be able to view the event queue.

In various embodiments, additional features to the clinical portal may include customization of event thresholds and notifications, mode switching, prescribed wear time slider functionality, and portal toggling for multiple devices in the single clinical portal.

While the invention has been particularly shown and described as referenced to the embodiments thereof, those skilled in the art will understand that the foregoing and other changes in form and detail may be made therein without departing from the spirit and scope.

What is claimed is:

1. A system for electrocardiography monitoring, comprising:
a wearable electrocardiography monitoring device; and
a clinical portal, wherein the clinical portal associates the wearable electrocardiography monitoring device with an individual user, and wherein the clinical portal presents (1) patient-specific information and (2) device-specific information to a clinician.

2. The system of claim 1, wherein the clinician activates the monitoring device via the clinical portal.

3. The system of claim 2, wherein the clinician deactivates the monitoring device via the clinical portal.

4. The system of claim 1, wherein the clinical portal presents the patient-specific information with information for a plurality of additional patients, each associated with a plurality of additional wearable electrocardiography monitoring devices.

5. The system of claim 1, wherein the clinical portal presents a total wear time, a start date, and end data, and a time remaining for the wearable electrocardiography monitoring device.

6. The system of claim 1, wherein the clinical portal presents clinician-identified cardiac events and patient-identified cardiac events.

7. The system of claim 1, wherein the clinical portal presents a device history of device-specific information for a former wearable electrocardiography monitoring device worn prior to the wearable electrocardiography monitoring device.

8. The system of claim 1, wherein, responsive to the wearable electrocardiography monitoring device reaching its total wear time, the clinical portal deactivates the monitoring device.

9. The system of claim 8, wherein, responsive to deactivation of the monitoring device, a deactivation prompt is sent to a mobile device associated with the wearable electrocardiography monitoring device.

10. The system of claim 1, wherein the clinical portal permits the clinician to view and modify at least one alert threshold for cardiac signals, the alert threshold corresponding to a potential heart problem.

11. The system of claim 1, wherein the clinical portal handshakes with the wearable electrocardiography monitoring device, prior to activation of the wearable electrocardiography monitoring device.

12. The system of claim 1, wherein the clinician can add doctor-specific notes and associate the doctor-specific notes with the wearable electrocardiography monitoring device.

13. The system of claim 1, further comprising a mobile device, wherein cardiac data measured by the wearable electrocardiography monitoring device is transmitted to the mobile device.

14. The system of claim 13, wherein cardiac data measured by the wearable electrocardiography monitoring device is transmitted from the mobile device to an external server.

15. The system of claim 14, wherein cardiac data measured by the wearable electrocardiography monitoring device is accessed at the external server by the clinical portal.

16. A system for electrocardiography monitoring, comprising:
a wearable electrocardiography monitoring device;
a mobile device, wherein cardiac data measured by the wearable electrocardiography monitoring device is transmitted to the mobile device; and
a clinical portal, wherein the clinical portal associates the wearable electrocardiography monitoring device with an individual user.

17. The system of claim 16, wherein the clinical portal presents (1) patient-specific information and (2) device-specific information to a clinician.

18. The system of claim 16, wherein a clinician activates the monitoring device via the clinical portal.

19. The system of claim 18, wherein the clinician deactivates the monitoring device via the clinical portal.

20. The system of claim 16, wherein the clinical portal presents a total wear time, a start date, and end data, and a time remaining for the wearable electrocardiography monitoring device.
